(19) United States
(12) Patent Application Publication
(10) Pub. No.: US 2007/0219162 A1
(43) Pub. Date: Sep. 20, 2007

(54) Partially-fluorinated ethers, compositions and uses thereof, for long-term tamponade in the eye

(75) Inventors: Janet R. Sparrow, New Rochelle, NY (US); Stanley Chang, Scarsdale, NY (US); Roderic N.F. Simpson, Monmouthshire (GB)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/605,688

(22) Filed: Nov. 29, 2006

Related U.S. Application Data
(63) Continuation of application No. PCT/US05/18921, filed on May 27, 2005.
(60) Provisional application No. 60/576,218, filed on Jun. 1, 2004.

(51) Int. Cl. A61K 31/075; A61K 31/02; A61K 31/695

(52) U.S. Cl. 514/63; 514/715; 514/743

(57) ABSTRACT

The present invention provides a single high-purity liquid comprising a partially fluorinated ether liquid for use as a long-term tamponade agent. Compositions are directed to liquid tamponade agents consisting essentially of DFPE, or to agents comprising a soluble mixture of a partially fluorinated ether and either a silicone liquid (or oil) or a perfluorocarbon liquid (or any other liquid or oil that is clear, colorless, inert and has a low specific gravity). Partially fluorinated ethers that have a specific gravity between 1.1 and 1.5 are well suited to rectify the deficiencies of silicone and perfluorocarbon liquids when they are used as tamponade agents. The soluble mixtures of the invention can be designed to have a specific gravity so that they do not have buoyancy problems (density greater than water), yet having a specific gravity that is not so high as to damage the posterior retina from mechanical forces (specific gravity less than 1.6). The mixtures can also be designed to have a specific gravity such that the mixture can act as a tamponade on both the superior and inferior retina simultaneously. The present invention provides such soluble mixtures and also partially fluorinated ether liquids alone, for the use and for the method of postoperative tamponade. Thus, partially fluorinated ether liquids, and mixtures thereof, can be left in the eye postoperatively due to their long-term compatibility with the eye, thereby improving the success of retinal reattachment and healing procedures.

This application is a continuation of PCT/US2005/018921 filed May 27, 2005, which claims priority to U.S. Ser. No. 60/576,218, filed Jun. 1, 2004, the disclosures of which are hereby incorporated by reference in their entirety.

This disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights.

All patent applications, published patent applications, issued and granted patents, texts, and literature references cited in this specification are hereby incorporated herein by reference in their entirety.

### BACKGROUND OF THE INVENTION

After surgery to repair retinal tears or detachments, surgeons often use postoperative tamponade agents to help maintain or support the surgical reattachment of the retina. By exerting a force that maintains the neural retina in apposition to the retinal pigment epithelium (RPE), postoperative tamponade agents help reattached retinas to heal. Examples of postoperative tamponade agents include intravitreal gases, silicone liquids (and oils) and perfluorocarbon liquids. However, these substances each possess limitations as a postoperative tamponade agent.

For example, intravitreal or intraocular gases (such as perfluoromethane, perfluoroethane, perfluoropropane, and sulphur hexafluoride) often require the patient to maintain a certain head position in order to maintain the pressure of the gas upon the reattached retina. This can be problematic for patients such as children or the elderly who cannot comply with head positioning. It is also a problem for patients who need to travel by air since rapid changes in atmospheric pressure can cause rapid enlargement of the gas bubble which increases intraocular pressure to damaging levels. Similarly, general inhalation anesthesia cannot be used for patients who require dental work or other surgical intervention because the high solubility of the inhalation gas (e.g. nitrous oxide) allows it to rapidly diffuse into the intraocular gas bubble causing increased bubble enlargment and increased intraocular pressure. Silicone liquids also have buoyancy problems. Since silicone liquids are lighter than water (specific gravity of 0.97), a silicone globule contacts the retina with a buoyant force directed upward, resulting in little real tamponade to the inferior retina with a patient sitting upright. To rectify this deficiency, attempts have been made to use fluorinated derivatives of silicone liquids because they have a density greater than water. However, fluorinated silicone liquids are associated with pronounced inflammatory responses, which can be due in part to the difficulty in isolating these liquids in high purity due to their composition of short chain polymers (Gabel, V.-P. et al. Br. J. Ophthalmol, 71:262 (1987)).

Perfluorocarbon liquids are also heavier than water (having a specific gravity greater than 1.6), thereby being a potential tamponade agent without presenting buoyancy and positioning problems. However, perfluorocarbon liquids are too heavy, the downward force of perfluorocarbon liquids for times greater than 1 week can result in the thinning of the outer plexiform layer of the inferior retina (Chang et al., Retina, 11:367-374 (1991)) or other significant negative effects. Thus, tamponade agents are desired that are heavier than water, lighter than perfluorocarbon liquids and are compatible with the eye for long-terms.

### SUMMARY OF THE INVENTION

The present invention is directed to partially fluorinated ethers that have a specific gravity of about 1.1 to about 1.5, such as decafluoro-di-n-pentyl ether (“DFPE”), for use primarily as a postoperative tamponade agent to aid retinal reattachment. In addition to having a specific gravity of about 1.1 to about 1.5, partially fluorinated ether liquids that are best-suited as a postoperative tamponade agent can have the following characteristics: inertness in the eye, available in very high purity, compatible with long-term residence in the eye, hydrophobic, optically clear and distinguishable from aqueous liquids, and low vapor pressure. Unlike prior tamponade agents, the partially fluorinated ether liquids of the invention can provide a sufficient force in the inferior and posterior direction of the eye such that it does not present buoyancy and positioning problems, while also not providing too much of a force such that the retina is damaged over a long-term postoperative presence in the eye. Further, the present invention provides soluble liquids comprising partially fluorinated ethers and silicone or perfluorocarbons (or other clear, colorless, and inert liquids that have a low specific gravity) for use as long-term tamponade agents that circumvent the problems of using silicone or perfluorocarbon liquids alone. The combination of partially fluorinated ethers with silicone and/or perfluorocarbon liquids (liquids in which partially fluorinated ethers are soluble) can also provide a postoperative tamponade agent that can facilitate support for the superior and inferior retina simultaneously.

In one aspect, the invention provides a liquid tamponade agent comprising decafluoro-di-n-pentyl ether (“DFPE”). In another aspect, the invention provides a liquid tamponade agent consisting essentially of DFPE. In one aspect, where a liquid tamponade agent consists essentially of DFPE, the agent can be greater than about 99%, 99.9%, 99.95% or 99.97% DFPE.

In one aspect, the invention provides a liquid tamponade agent comprising a soluble mixture of a partially fluorinated ether liquid and any other liquid(s) or oil(s) (or combinations of other liquid(s) and oil(s)) that is inert, clear, colorless and has a low specific gravity. For example, the liquid or oil (or combinations thereof) that is inert, clear, and colorless can have a specific gravity that is from about 0.85 to about 1 (or to about less than 1), from about 0.9 to about 1 (or to about less than 1), from about 0.95 to about 1 (or to about less than 1), or from about 0.96 to about 0.99, or from about 0.96 to about 0.98, or at about 0.97. In one aspect, the invention provides a liquid tamponade agent comprising a soluble mixture of a partially fluorinated ether liquid and a silicone oil. In another aspect, the invention provides a liquid tamponade agent comprising a soluble mixture of a partially fluorinated ether liquid and a perfluorocarbon liquid.

In respect to the liquid tamponade agents, the partially fluorinated ether liquid comprises a fluorinated ether having the formula (I): Rf(CH₂)ₙO(CH₂)ₙRf, wherein Rf is a C₁₋₄ fluorinated monovalent saturated organic group, and n is 3 or 4. In one aspect, Rf is a C₁₋₄ polyfluoroalkyl group, a C₁₋₄ perfluoroalkyl group, or —C₂F₅; in a further aspect, n is 3.

In another aspect, the liquid tamponade agents of the present invention have a specific gravity greater than 1 and less than 1.6. In another aspect, the liquid tamponade agents have a specific gravity of about 1.2 or about 1.3. In another aspect, the liquid tamponade agents have a specific gravity of about 1.02 to about 1.03.

In another aspect, the soluble mixture of a liquid tamponade agent comprises decafluoro-di-n-pentyl ether (C₂F₅CH₂CH₂CH₂OCH₂CH₂CH₂C₂F₅) (“DFPE”).

In another aspect, a liquid tamponade agent comprises a soluble mixture of DFPE and silicone oil, wherein the soluble mixture comprises between about 10% and about 20% of DFPE and between about 90% and about 80% silicone oil. In another aspect, a liquid tamponade agent comprises a soluble mixture of DFPE and silicone oil, wherein the soluble mixture comprises between about 15% and about 18% of DFPE and between about 85% and about 82% silicone oil. In another aspect, a liquid tamponade agent comprises a soluble mixture of DFPE and silicone oil, wherein the soluble mixture comprises about 20% DFPE and about 80% silicone oil. In another aspect, the liquid tamponade agent comprising a soluble mixture of DFPE and silicone oil has a density greater than water. In one aspect, such a mixture has a specific gravity between about 1.00 and about 1.06. In one aspect, such a mixture has a specific gravity between about 1.02 and about 1.03.

In another aspect, a liquid tamponade agent comprises a soluble mixture of a partially fluorinated ether and a perfluorocarbon liquid. The perfluorocarbon liquid can be, for example, perfluoro-n-octane (“PFnO”), perfluorophenanthrene, perfluorodecalin or perfluoroethylcyclohexane. In another aspect, the liquid tamponade agents comprising a soluble mixture of a partially fluorinated ether and a perfluorocarbon liquid have a specific gravity less than 1.6.

The present invention also provides for the use of any of the liquid tamponade agents disclosed herein as a postoperative tamponade agent, wherein the operation was for the treatment of an eye disease, eye disorder or eye damage. This includes the use of the liquid tamponade agents comprising a soluble mixture, as mentioned above, and the use of a partially fluorinated ether liquid alone, wherein the partially fluorinated ether liquid comprises a fluorinated ether having the formula (I): Rf(CH₂)ₙO(CH₂)ₙRf, wherein Rf is a C₁₋₄ fluorinated monovalent saturated organic group, and n is 3 or 4. In other aspects, Rf can be a C₁₋₄ polyfluoroalkyl group, a C₁₋₄ perfluoroalkyl group or —C₂F₅. In another aspect, n is 3. In another aspect, the invention provides for the use of DFPE alone or “neat” as a tamponade agent.

In the present invention, the use or methods of postoperative tamponades can be after an operation for the treatment of an eye disorder, eye disease or eye damage that can be, for example, proliferative vitreoretinopathy, complicated diabetic retinal detachment, giant retinal tears, traumatic retinal detachment, subretinal hemorrhage, vitreous hemorrhage, dislocation of lens fragments or lens implant, retinal detachment secondary to macular hole, or after removal of a foreign object or repair of ocular trauma.

In another aspect, the partially fluorinated ether liquid for use as a postoperative tamponade agent is DFPE. In another aspect, the partially fluorinated ether liquid for use as a postoperative tamponade agent comprises the following characteristics: available in very high purity, hydrophobic, optically clear, distinguishable from aqueous liquids, low vapor pressure, and a specific gravity of between about 1I.1 and about 1.5. In one aspect, the partially fluorinated ether liquid has a purity that is greater than about 99.9%. In another aspect, the purity is greater than about 99.95%. In another aspect, the purity is greater than about 99.97%. In another aspect, the partially fluorinated ether liquid further comprises the characteristic of being inert in the eye.

In another aspect, the present invention provides a tamponade agent (either a soluble mixture as described above or a partially fluorinated ether alone) that can be left in the eye postoperatively for a time period greater than 1 day, 1 week, 2 weeks, 1 month, 2 months, 6 months, up to about 1 year, up to about 2 years or more, without having a significant negative effect on the eye. By significant negative effect, one skilled in the art understands that some degree of negative effects are acceptable, as minor negative effects are balanced in view of retinal repair. In one aspect, the negative effect on the eye comprises a thinning of the outer plexiform layer in the inferior retina. In other aspects, the negative effect can comprise one or more of the following indications in the dependent retina: displacement of photoreceptor nucleic into the rod and cone layer (photoreceptor drop down), distortion of photoreceptor outer segments, narrowing of the outer plexiform layer, and retinal pigment epithelial cell hypertrophy. Additionally, where the present invention provides a tamponade agent that has a simultaneous force on the superior and inferior retina, then the tamponade agent can be used for long-terms postoperatively without negative indications (analogous to the ones described above) in the superior retina.

In another aspect, the present invention provides a method for postoperative tamponade in the eye comprising administering into the vitreous chamber of the eye an effective amount of a liquid tamponade agent. The liquid tamponade agent can be a soluble mixture as described above, or the agent can be simply a partially fluorinated ether liquid, also as described above. Thus, in one aspect, a method of postoperative tamponade in the eye comprises administering into the vitreous chamber of the eye an effective amount of a liquid tamponade agent, wherein the liquid tamponade agent is a partially fluorinated ether liquid, and wherein the partially fluorinated ether liquid comprises a fluorinated ether having the formula (I): Rf(CH₂)ₙO(CH₂)ₙRf, wherein Rf is a C₁₋₄ fluorinated monovalent saturated organic group, and n is 3 or 4. In other aspects, Rf can be a C₁₋₄ polyfluoroalkyl group, a C₁₋₄ perfluoroalkyl group or Rf is —C₂F₅. In another aspect, n is 3. In another aspect, the partially fluorinated ether liquid is DFPE.

In the methods of postoperative tamponade, the tamponade can be after an operation that was for the treatment of an eye disorder, an eye disease or eye damage. The eye disorder, eye disease or eye damage can be, for example, proliferative vitreoretinopathy, complicated diabetic retinal detachment, giant retinal tears, traumatic retinal detachment, subretinal hemorrhage, vitreous hemorrhage, dislocation of lens fragments or lens implant, retinal detachment secondary to macular hole, or after removal of a foreign object or repair of ocular trauma.

In another aspect of the methods of postoperative tamponade, wherein tamponade agent is a partially fluorinated ether liquid, the partially fluorinated ether liquid comprises the following characteristics: available in very high purity, hydrophobic, optically clear, distinguishable from aqueous liquids, low vapor pressure, and a specific gravity of between about 1.1 and about 1.5. In one aspect, the purity is greater than about 99.9%. In another aspect, the purity is great than about 99.95%. In another aspect, the purity is great than about 99.97%. In another aspect, the partially fluorinated ether liquid further comprises the characteristic of being inert in the eye.

In another aspect of the methods of postoperative tamponade, the tamponade agent can be left in the eye postoperatively for a time period greater than 1 day, 1 week, 2 weeks, 1 month, 2 months, 6 months, 1 year, or up to about 2 years (or more) without having a negative effect on the eye. In one aspect, the negative effect on the eye comprises a thinning of the outer nuclear layer or outer plexiform layer in the inferior retina.

In another aspect of the present methods, the liquid tamponade agent maintains the neural retina in apposition to the retinal pigment epithelium (RPE).

In another aspect, the invention provides the use of a partially fluorinated ether liquid or a soluble mixture of a partially fluorinated ether liquid and a perfluorocarbon liquid and/or a silicone liquid in combination with an intraocular gas for postoperative tamponade. The intraocular gas can be, for example, a perfluorocarbon gas. The perfluorocarbon gas can be, for example, perfluoromethane, perfluoroethane, perfluoropropane or sulphur hexafluoride.

In another aspect, the present invention provides a method or use of a perfluorocarbon liquid to remove a partially fluorinated ether tamponade agent. In this aspect, a perfluorocarbon that has a higher volatility than the partially fluorinated tamponade agent and a low viscosity can be used to wash out and remove trace partially fluorinated ether from the eye, as after washing, any trace perfluorocarbon liquid remaining in the eye will diffuse out of the eye more readily than trace partially fluorinated ethers due to vapor pressure.

In another aspect of the present invention, partially fluorinated ether liquids can be used as an intraoperative liquid tool to help manipulate retinal tears, breaks, holes or folds, and can be compatible with the use of laser photocoagulation.

### BRIEF DESCRIPTION OF THE DRAWINGS

FIG. 1 depicts the internal structures of the eye through a saggital section.

FIG. 2 depicts additional structures of the eye. The top of the figure corresponds with the anterior end of the eye (towards the cornea) and the bottom of the figure corresponds with the posterior end of the eye (towards the optic nerve).

FIGS. 3A and 3B both depict a retinal detachment. When the retina detaches, it separates from the back wall of the eye and loses its blood supply and source of nutrition and its proximity to the retinal pigment epithelium. The retina will degenerate and lose its ability to function if it remains detached. Central vision will be lost if the macula remains detached. Retinal detachment can be divided into three main categories: (1) Rhegmatogenous retinal detachment, which is due to a retinal break or tear that allows the liquid vitreous to pass through the break and detach the retina; (2) Exudative retinal detachment, which is due to leakage from under the retina which creates fluid (exudates) that detaches the retina—tumors and inflammatory disorders can create exudative detachments; and (3) Traction retinal detachments, which are due to pulling on the retina usually from fibro-vascular tissue within the vitreous cavity—proliferative diabetic retinopathy is a common cause of traction retinal detachments.

FIG. 4 and FIG. 5 show results indicating the long term biocompatibility of the eye to decafluoro-di-n-pentyl ether. Retinal tolerance to DFPE is manifested by the absence of adverse effects over long terms. For example, FIGS. 4 and 5 show that after 3 months in the eye, DFPE does not exhibit any negative effects as shown by the implicit times (FIG. 4) and amplitudes (FIG. 5) of electroretinographic responses. Further, the DFPE tamponade liquid bubble remained coherent, did not show any tendency to emulsify or break into droplets, and did not show any cloudiness as would have been the result of an adverse physiological reaction. Eyes with DFPE did not exhibit any differences in eye function as compared to eyes with a balanced salt solution (BSS).

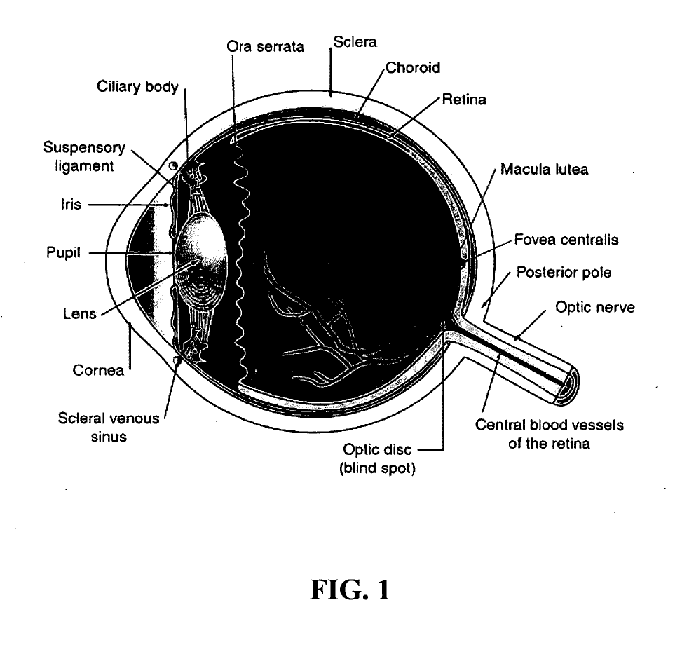

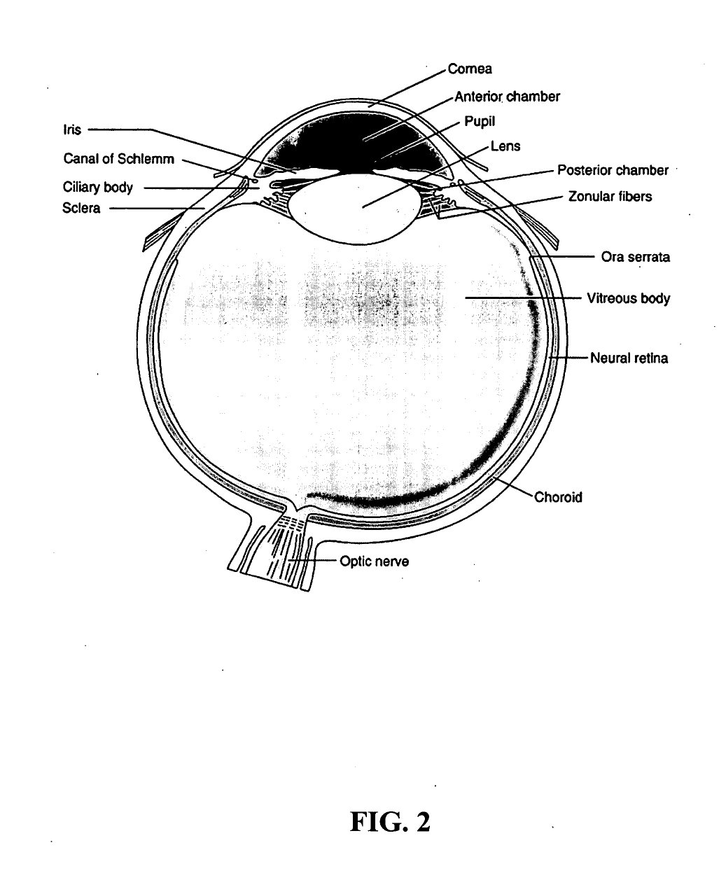

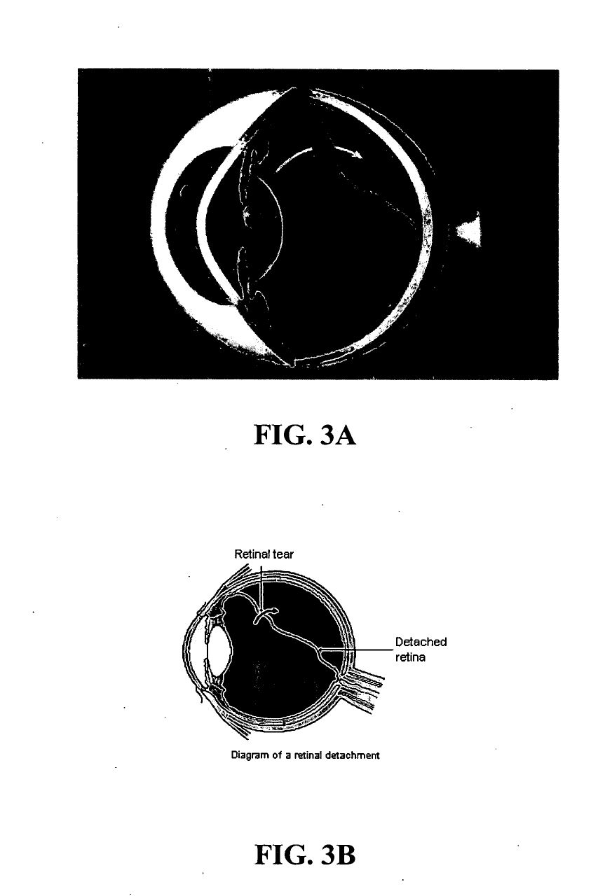

### DETAILED DESCRIPTION OF THE INVENTION

The present invention provides compositions, uses and methods directed to partially fluorinated ether liquids as a long-term postoperative tamponade agent. The compositions are directed to liquid tamponade agents comprising a soluble mixture of a partially fluorinated ether and a clear, colorless, inert liquid having a low specific gravity (such as a silicone liquid (or oil) and a perfluorocarbon liquid). The compositions are also directed to liquid tamponade agents comprising of or consisting essentially of DFPE. Partially fluorinated ethers that have a specific gravity between 1.1 and 1.5 are well suited to rectify the deficiencies of silicone and perfluorocarbon liquids when they are used as tamponade agents. The soluble mixtures of the invention can be designed to have a specific gravity so that they do not have buoyancy problems (density greater than water), yet having a specific gravity that is not so high as to damage the posterior retina from mechanical forces (specific gravity less than 1.6). The mixtures can also be designed to have a specific gravity such that the mixture can act as a tamponade on both the superior and inferior retina simultaneously. The present invention provides such soluble mixtures and also partially fluorinated ether liquids alone, for the use and for the method of postoperative tamponade. Thus, partially fluorinated ether liquids, and mixtures thereof, can be left in the eye postoperatively due to their long-term compatibility with the eye, thereby improving the success of retinal reattachment and healing procedures.

Characteristics of partially fluorinated ether liquids. The partially fluorinated ether liquids contemplated in the present invention are generically described by formula (I): Rf(CH₂)ₙO(CH₂)ₙRf, wherein Rf is a C₁₋₄ fluorinated monovalent saturated organic group, and n is 3 or 4. Rf can be, for example, a C₁₋₄ polyfluoroalkyl group, a C₁₋₄ perfluoroalkyl group, or C₂F₅, where n is 3. The Rf group may be linear or branched. The Rf group comprises carbon atoms and fluorine atoms, as Rf is a perfluorinated carbon structure. Examples of specific partially fluorinated ether liquids for use in the invention are: DFPE (decafluoro-di-n-pentyl ether; C₂F₅CH₂CH₂CH₂OCH₂CH₂CH₂C₂F₅); CF₃CH₂CH₂CH₂OCH₂CH₂CH₂CF₃; and (CF₃)₂CFCH₂CH₂CH₂OCH₂CH₂CH₂CF(CF₃)₂. The fluorinated ethers (and methods for making these ethers) contemplated in the present invention are also described in International Patent Application PCT/JP03/15279.

It is desired for Rf to contain not more than four carbons in order to keep the specific gravity of the partially fluorinated ether liquid below about 1.5. When the specific gravity of a liquid tamponade agent exceeds 1.5, then the probability of damage to the posterior retina increases due to long-term mechanical (posterior) forces exerted by the heavy tamponade agent. Thus, the use of partially fluorinated ether liquids of formula (I) are advantageous over perfluorocarbon liquids because perfluorocarbon liquids have a specific gravity greater than 1.6. The reduced weight of partially fluorinated ether liquids of formula (I) makes it less likely to damage the retina due to pressure over the long-term.

Additionally, partially fluorinated ether liquids described by formula (I) have a density greater than water (the specific gravity range of the partially fluorinated ether liquids of formula (I) are between about 1.1 and about 1.5). This is important because tamponade agents (liquids, oils or gases) that are lighter than water present buoyancy problems in the eye. These lighter agents rise toward the superior end of the eye when a patient is in an upright position. Thus, if tamponade agents that are lighter than water are used to assist the postoperative healing of posterior retinal damage, then the patient must maintain awkward head positions such that the buoyant force of the agent can be directed posteriorly. However, such awkward head positions are difficult for certain patients to maintain, such as children or elderly people. Further, intravitreal gases are also problematic for patients who need to travel by air since rapid changes in atmospheric pressure can cause rapid enlargement of the gas bubble which increases intraocular pressure to damaging levels. Similarly, general inhalation anesthesia cannot be used for patients who require dental work or other surgical intervention because the high solubility of the inhalation gas (e.g., nitrous oxide) allows it to rapidly diffuse into the intraocular gas bubble causing increased bubble enlargement and increased intraocular pressure.

Further, silicone liquids are lighter than water (specific gravity of 0.97), thus, a silicone globule contacts the retina with a buoyant force directed upward, resulting in little real tamponade to the inferior and posterior retina with a patient sitting upright. To rectify this deficiency, attempts have been made to use fluorinated derivatives of silicone liquids because they have a density greater than water. However, fluorinated silicone liquids are associated with pronounced inflammatory responses, which can be due to the difficulty in isolating these liquids in high purity due to their composition of short chain polymers. This difficulty is further enhanced by the fact that these short chain polymers are difficult to characterize, and as such, may conceal impurities.

Other advantageous characteristics of partially fluorinated ether liquids for use as a long-term postoperative tamponade agent include: available in very high purity, hydrophobic (almost to the same extent as perfluorocarbons), optically clear, distinguishable from aqueous liquids, low vapor pressure, low viscosity and inertness in the eye. By very high purity, the present invention means that partially fluorinated ether liquids can be isolated at purity greater than about 99.9%. In other embodiments, partially fluorinated ethers can be isolated at purity greater than about 99.95% or about 99.97%. Analysis of partially fluorinated ethers can be conducted with nuclear magnetic resonance spectroscopy, gas chromatography and mass-spectrometry, for example. The characteristics of hydrophobicity and optical distinctiveness from aqueous liquids make partially fluorinated ether liquids easy to remove from the eye. Further, being optically clear, the use of partially fluorinated ethers will not impede vision. In addition to its lighter weight (than perfluorocarbon liquids), partially fluorinated ethers can be further compatible with the eye for long time periods due its characteristic of being substantially inert in the eye.

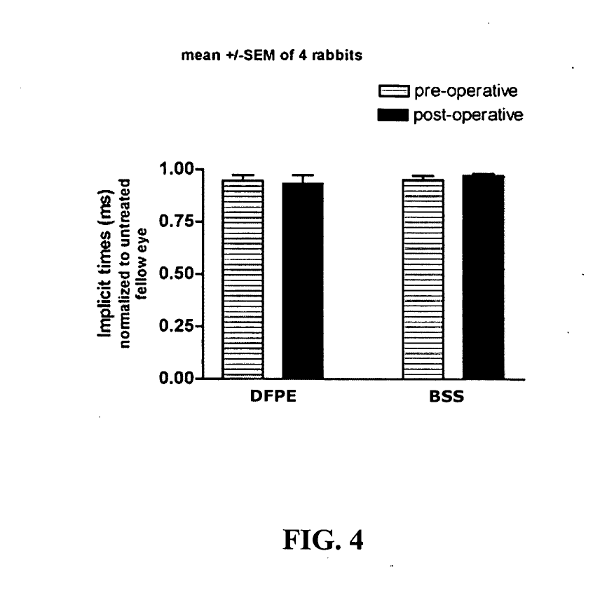

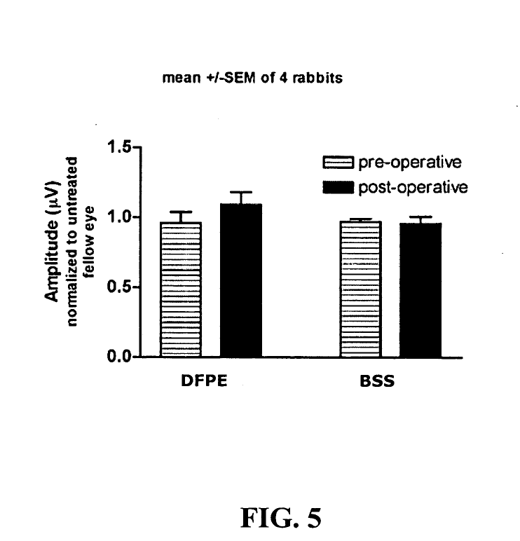

Thus, the above characteristics, combined with the specific-gravity characteristics of the partially fluorinated ethers of formula (I), allow for their long-term use in the eye without damage, and with improved efficacy, convenience and biocompatibility. For example, studies of retinal tolerance to decafluoro-di-n-pentyl ether (which has a specific gravity of 1.31 at body temperature) have shown no adverse effects for long terms. FIGS. 4 and 5 show that after 3 months in the eye, DFPE does not exhibit any negative effects as manifested by the implicit times and amplitudes of electroretinographic responses. Further, neither was there any deterioration of the DFPE tamponade bubble in the eyes, such as cloudiness (which is also an indication of an adverse physiological reaction), emulsification or breakage into smaller droplets. The data shows no inferior responses in eyes with DFPE as compared to eyes with balanced salt solution. The invention contemplates the use of liquid tamponade agents comprising partially fluorinated ethers according to formula (I) for postoperative periods such as: hours, days, weeks, 1 month, 2 months, 3 months, greater than 3 months, greater than 6 months, up to 1 year, up to 2 years or more. Period greater than 1 year are not generally required for tamponade. However, the invention does not necessarily preclude the use of partially fluorinated ethers of formula (I) as long-term postoperative tamponade agents for periods greater than 1 year.

DFPE liquid is stable. DFPE liquid is stable over time at ambient temperature (at least 2 years), it is stable when exposed to heat up to 95° C., and is stable to sunlight exposure (for example, up to a week when stored in a clear glass container). Further, a mixture of DFPE in silicone (20% DFPE (w/w) and 80% silicone (w/w)) is stable through daily temperature cycling (20-40° C.) through at least 18 months. Further with respect to water insolubility, a single drop of DFPE (about 50 ppm) added to one liter of water remains visible, unchanged and undissolved for long periods of time (at least greater than 8 months). Further, from analysis by gas chromatography, DFPE recovered from the eye after one month of residence time does not show any significant alterations, as changes in composition of DFPE were less than 0.05%, which was probably due to its contact with various external materials such as syringes and filters prior to its residence in the eye.

The characteristics of partially fluorinated ethers also enable their use during retinal surgery. However, perfluorocarbon liquids have a greater flattening force on detached retinas due to their greater weight, and as such, partially fluorinated ether liquids are generally best-suited and most-advantageous for postoperative tamponade. Thus, the present invention provides the use of partially fluorinated ether liquids as long-term postoperative tamponade agents in the eye, as these ether liquids are more effective than prior tamponade agents like intravitreal gases, silicone liquids (and oils) and perfluorocarbon liquids.

Table 1 below lists some physical and chemical properties of various tamponading agents (the tamponade force is calculated by the formula: tamponade force=the difference in specific gravity between the tamponade agent and the fluid vitreous, where the fluid vitreous has a specific gravity of 1.03, multiplied by the volume of the tamponade agent (5 mL)):

TABLE 1
Physical and Chemical Properties of Tamponading Agents

| | | | | | Silicone oil | | 25% PFnO |
|---|---|---|---|---|---|---|---|
| | | Perfluoro- | Perfluoro- | | (dimethyl | | and 75% |
| | PFnO | decalin | phenanthrene | Healon | siloxane) | DFPE | DFPE |
| Specific | 1.76 | 1.94 | 2.03 | 1.036 | 0.97 | 1.31 | 1.39 |
| Gravity | | | | | | | |
| Boiling | 105° C. | 177° C. | 215° C. | 100° C. | >200° C. | ˜200° C. | |
| Point | | | | | | | |
| Viscosity | 0.8 | 2.7 | 8.03 | 200,000 | 1000-5000 | similar to | |
| (centistrokes | | | | | | perfluoro- | |
| at 25° C.) | | | | | | decalin | |
| Refractive | 1.27 | 1.31 | 1.28 | 1.335 | 1.40 | similar to | |
| Index | | | | | | perfluoro- | |
| | | | | | | decalin | |
| Tamponade | 3.65 | 4.55 | 5.0 | 0.03 | 0.3 | 1.4 | 1.8 |
| Force | | | | | | | |

Soluble Mixtures of Partially Fluorinated Ethers. The presence of the hydrocarbon chain in the structure of formula (I) has a large effect on the solubility properties of the molecule. The partially fluorinated ethers of formula (I) are completely miscible with certain perfluorocarbons, such as perfluoro-n-octane (“PFnO”) (C₈F₁₈), perfluoroethylcyclohexane (C₈F₁₆), perfluorodecalin (C₁₀F₁₈), and perfluorophenanthrene (C₁₄F₂₄). The solubility of formula (I) liquids with perfluorocarbons enables the design of soluble mixtures that can be used as long-term postoperative tamponade agents that have a specific gravity less than 1.6 or 1.5 such that the probability of retinal damage due to weight is reduced. For example, the commonly used intraoperative perfluorocarbon, PFnO, has a specific gravity of about 1.7. Thus, in order to make a soluble mixture of PFnO and DFPE (which has a specific gravity of about 1.3) that has a specific gravity less than 1.6 or 1.5, then one can use a greater amount of DFPE to reduce the specific gravity of the soluble mixture. For example, a mixture comprising about 25% PFnO and about 75% DFPE has a specific gravity of 1.39 at body temperature (see Table 1). This general strategy of mixing lighter partially fluorinated ether liquids with perfluorocarbon liquids can be conducted with any of the formula (I) liquids and a perfluorocarbon liquid, as long as they are miscible. Further, one can also make soluble mixtures between different partially fluorinated ethers in order to obtain a liquid tamponade agent that has a preferred specific gravity.

Further, one can use the strategy of mixing partially fluorinated ether liquids of formula (I) with lighter tamponade liquids, such as silicone, in order to resolve buoyancy problems and to increase tamponade force. As described previously, because silicone liquids are lighter than water and are immiscible in water, silicone liquids form a globule that rises above aqueous solutions. To rectify this problem, partially fluorinated ether liquids can be mixed with silicone liquids and oils such that soluble mixture has a specific gravity greater than 1.0. In one embodiment, partially fluorinated ether and silicone solutions have a specific gravity between about 1.00 and about 1.06. For example, DFPE can be mixed with silicone oil (such as dimethyl siloxane) and be soluble in silicone oil up to about 20% DFPE (weight to weight (w/w)). Thus, the invention contemplates DFPE and silicone mixtures where DFPE comprises between about 10% and about 20% of the solution and where silicone comprises between about 90% and about 80% of the solution. In one embodiment, a soluble mixture comprises between about 15% and about 18% (w/w) DFPE and between about 85% and about 82% (w/w) silicone oil. In another embodiment, a soluble mixture of about 18% (w/w) DFPE and about 82% (w/w) silicone oil has a specific gravity of about 1.02 at 37° C. In another embodiment, a soluble mixture of about 20% (w/w) DFPE and about 80% (w/w) silicone oil has a specific gravity of about 1.02-1.03. Perfluorocarbon liquids cannot be easily mixed with silicone liquids because perfluorocarbon liquids, not having any hydrocarbons, are only soluble up to less than about 1000 ppm with silicone.

Additionally, partially fluorinated ether liquids can be mixed with perfluorocarbon liquids or silicone liquids in order to design tamponade agents that exhibit tamponade forces in the superior, inferior and posterior directions simultaneously. The invention also provides that partially fluorinated ether liquids can be mixed with essentially any liquid or oil (or mixed with combinations of more than one liquid, of more than one oil, or of a liquid and an oil, or of more than one liquid and more than one oil) that is clear, colorless, inert and has a low specific gravity (i.e., from about 0.85 to about 1 or less than 1, for example) such that the mixture exhibits tamponade forces in the superior, inferior and posterior directions simulataneously.

Further, the present invention also encompasses the use of partially fluorinated ether liquids (and soluble mixtures thereof) in combination with intravitreal gases for postoperative tamponade. In one embodiment, the invention provides for the use of partially fluorinated ether liquids (and soluble mixtures thereof) in combination with perfluorocarbon gases, such as perfluoromethane, perfluoroethane, perfluoropropane and sulfur hexafluoride, as a postoperative tamponade.

Indications for use and techniques. In the present methods and uses of postoperative tamponade, the tamponade can be after an operation that was for the treatment of an eye disorder, an eye disease or eye damage. The eye disorder, eye disease or eye damage can be, for example, proliferative vitreoretinopathy, complicated diabetic retinal detachment, giant retinal tears, traumatic retinal detachment, subretinal hemorrhage, vitreous hemorrhage, presence of a foreign object, dislocation of lens fragments or lens implant, retinal detachment secondary to macular hole or ocular trauma, or after removal of a foreign object or repair of ocular trauma (for a description of intraoperative treatments to these disorders, see Retina-Vitreous-Macula, Editors Guyer, D. et al., W.B. Saunders Company, 1999, in the chapter “Vitreous Substitutes,” by Chang and Sparrow, pages 1320-1337). The liquid tamponade agents of the present invention are placed into the eye with the same standard techniques used to introduce perfluorocarbon liquids into the eye (for general techniques, see U.S. Pat. No. 4,490,351; U.S. Pat. No. 5,037,384; Bourke, R. and Cooling, R, Australian and New Zealand J. Ophthalmol., 1995, 23(3):165-171; and Comaratta, M. and Chang, S., Curr. Opinion Ophthalmol., 1991, 2:291-298).

As for the removal of liquid tamponade agents of the invention, rinsing the vitreous cavity with PFnO (or other perfluorocarbon liquid that has a higher volatility and low viscosity) can facilitate removal of partially fluorinated ether liquids from the eye. After removal of the majority of the fluorinated ether liquid from the vitreous cavity using standard techniques, trace fluorinated ethers in the eye can be removed by rinsing the eye with PFnO. Any residual or trace PFnO left in the eye after washing will diffuse away more quickly than partially fluorinated ethers because PFnO has a higher volatility.

Compositions and Kits. The liquid tamponade agents of the present invention, including partially fluorinated ethers alone and soluble mixtures thereof, can be used without any other ingredients or preservatives. The present invention also contemplates commercial kits that comprise, for example, a high purity partially fluorinated ether, or a high purity soluble mixture comprising a partially fluorinated ether in a vial (5 ml vial, 10 ml vial, 15 ml vial, etc.). In one embodiment, high purity can mean greater than 99.9% purity of the partially fluorinated ether or of the soluble mixture. The kit can further comprise a microbial filter unit, a 10 cc syringe, a beveled needle (20 gauge×1½″ beveled needle, for example), and a blunt cannula (23 gauge, for example). All kit components are provided sterile, and can be sterilized, for example, by either filtration or heating.

As various changes can be made in the above methods, uses and compositions without departing from the scope and spirit of the invention as described, it is intended that all subject matter contained in the above description, shown in the accompanying drawings, or defined in the appended claims be interpreted as illustrative, and not in a limiting sense.

### EXAMPLES

The example described below is provided to illustrate certain aspects of the present invention and is not included for the purpose of limiting the invention. It should be appreciated that those of skill in the art, in light of the present disclosure, will recognize that numerous modifications can be made without departing from the spirit and intended scope of the invention.

### Example 1

### Use of DFPE as a Postoperative Tamponade

The partially fluorinated ether liquid decafluoro-di-n-pentyl ether can be used as a vitreous substitute in conjunction with vitrectomy. Vitrectomy is a microsurgical procedure in which specialized instruments and techniques are used to repair retinal disorders. The initial step in this procedure is usually the removal of the vitreous liquid (or “gel”) through very small (˜1.4 mm) incisions in the eye wall. The surgeon can use a specialized operating microscope and contact lenses, which allow a clear view of the vitreous cavity and retina at various magnifications. A high intensity fiberoptic light source is used to illuminate the inside of the eye while the surgeon works. The vitreous gel is removed with a miniature handheld cutting device and simultaneous aspiration. In the next stage perfluorocarbon liquid is used as a soft tool to flatten, manipulate and re-position the retina. The perfluorocarbon liquid is then removed and the decafluoro-di-n-pentyl ether liquid for use as a postoperative tamponade agent would then be introduced to the vitreous cavity in a procedure such as fluid-fluid exchange or fluid-air-fluid exchange.

The use of partially fluorinated ether liquids as a vitreous substitute can be in combination with other vitreous substitutes, including perfluorocarbon liquids or silicone liquids. As partially fluorinated ether liquids are optically clear, they will not obscure the surgeon's field of operation. The surgeon can use endophotocoagulation (the use of lasers to treat intraocular structures) to treat retina tears in the setting of retinal detachment or proliferative diabetic retinopathy. Also, microsurgical instruments such as forceps, scissors and picks can be used to manipulate intraocular structures such as in the removal of scar tissue and foreign bodies.

What is claimed:

1. A liquid tamponade agent comprising a soluble mixture of a partially fluorinated ether liquid and another liquid or oil, wherein the another liquid or oil is clear, colorless, inert, and has a specific gravity from about 0.85 to about less than 1.0.

2. The liquid tamponade agent of claim 1, wherein the oil comprises a silicone oil.

3. The liquid tamponade agent of claim 1, wherein the another liquid comprises a perfluorocarbon liquid.

4. The liquid tamponade agent of claim 1, wherein the partially fluorinated ether liquid comprises a fluorinated ether having the formula (I): R^{f}(CH₂)ₙO(CH₂)ₙR^{f}, wherein R^{f} is a C₁₋₄ fluorinated monovalent saturated organic group, and n is 3 or 4.

5. The liquid tamponade agent of claim 4, wherein R^{f} is a C₁₋₄ polyfluoroalkyl group, a C₁₋₄ perfluoroalkyl group, or —C₂F₅.

6. The liquid tamponade agent of claim 5, wherein n is 3.

7. The liquid tamponade agent of claim 1, wherein the soluble mixture has a specific gravity greater than 1 and less than 1.6.

8. The liquid tamponade agent of claim 1, wherein the partially fluorinated ether liquid is decafluoro-di-n-pentyl ether (C₂F₅CH₂CH₂CH₂OCH₂CH₂CH₂C₂F₅).

9. The liquid tamponade agent of claim 2, wherein the partially fluorinated ether liquid is decafluoro-di-n-pentyl ether, and wherein the soluble mixture comprises between about 10% and about 20% of decafluoro-di-n-pentyl ether and between about 90% and about 80% silicone oil.

10. The liquid tamponade agent of claim 9, wherein the liquid tamponade agent has a density greater than water.

11. The liquid tamponade agent of claim 3, wherein the perfluorocarbon liquid comprises perfluoro-n-octane, perfluorophenanthrene, perfluorodecalin or perfluoroethylcyclohexane.

12. A liquid tamponade agent consisting essentially of decafluoro-di-n-pentyl ether.

13. A method for postoperative tamponade in the eye comprising administering into the vitreous chamber of the eye an effective amount of a liquid tamponade agent of claim 1.

14. The method of claim 13, wherein the partially fluorinated ether liquid comprises a fluorinated ether having the formula (I): R^{f}(CH₂)ₙO(CH₂)ₙR^{f}, wherein R^{f} is a C₁₋₄ fluorinated monovalent saturated organic group, and n is 3 or 4.

15. The method of claim 13, wherein the operation was for the treatment of an eye disorder, an eye disease or eye damage.

16. The method of claim 15, wherein the eye disorder, eye disease or eye damage comprises: proliferative vitreoretinopathy, complicated diabetic retinal detachment, giant retinal tears, traumatic retinal detachment, subretinal hemorrhage, vitreous hemorrhage, dislocation of lens fragments or lens implant, retinal detachment secondary to macular hole, or after removal of a foreign object or repair of ocular trauma.

17. The method of claim 14, wherein R^{f} is a C₁₋₄ polyfluoroalkyl group, a C₁₋₄ perfluoroalkyl group or R^{f} is —C₂F₅.

18. The method of claim 17, wherein n is 3.

19. The method of claim 14, wherein the partially fluorinated ether liquid is decafluoro-di-n-pentyl ether (C₂F₅CH₂CH₂CH₂OCH₂CH₂CH₂C₂F₅).

20. The method of claim 14, wherein the partially fluorinated ether liquid comprises the following characteristics: available in very high purity, hydrophobic, optically clear, distinguishable from aqueous liquids, low vapor pressure, and a specific gravity of between about 1.1 and about 1.4.

21. The method of claim 14, wherein the purity is greater than about 99.9%.

22. The method of claim 14, wherein the purity is greater than about 99.95%.

23. The method of claim 20, wherein the partially fluorinated ether liquid further comprises the characteristic of being inert in the eye.

24. The method of claim 13, wherein the tamponade agent can be left in the eye post-operatively for a time period greater than 2 months without having a negative effect on the eye.

25. The method of claim 13, wherein the tamponade agent can be left in the eye post-operatively for a time period up to about 2 years without having a negative effect on the eye.

26. The method of claim 24 or claim 25, wherein the negative effect on the eye comprises a thinning of the outer nuclear or outer plexiform layer in the inferior retina.

27. The method of claim 13, wherein the liquid tamponade agent maintains the neural retina in apposition to the retinal pigment epithelium (RPE).
